(19) United States
(12) Patent Application Publication
(10) Pub. No.: US 2002/0042658 A1
(43) Pub. Date: Apr. 11, 2002

(54) Hernia repair mesh prosthesis, and method of using same

(76) Inventor: Narendra S. Tyagi, Bloomfield Hills, MI (US)

Correspondence Address: CARRIER BLACKMAN AND ASSOCIATES, 24101 NOVI ROAD, SUITE 100, NOVI, MI, 48375

(21) Appl. No.: 09/974,261

(22) Filed: Oct. 9, 2001

Related U.S. Application Data
(60) Provisional application No. 60/239,212, filed on Oct. 10, 2000.

(51) Int. Cl. A61F 2/02

(52) U.S. Cl. 623/23.72; 623/23.75; 623/23.76; 606/151

(57) ABSTRACT

A hernia repair prosthetic element includes: a central portion which covers a hernia opening within a patient; plural support portions integrally connected about a periphery of the central portion for securing the central portion in covering relation to the hernia opening; the prosthetic element, including the central and support portions, being a substantially planar member formed of flexible mesh material; the support portions being foldable relative to the central portion so that the prosthetic element can be selectively manipulated between folded and unfolded shapes; the support portions being adapted to securely engage various components of the patient's body surrounding the hernia opening; and openings are formed through the central portion for passage of fluid therethrough. The prosthetic element has only two basic shapes, one for use in relation to all groin hernias and one for use in relation to all abdominal hernias. The flexible mesh material may be of propylene or other suitable materials, the central portion has an enhanced strength, such as being formed of two layers of the mesh material, and the central portion may be stretchable to absorb greater forces imposed thereon.

### BACKGROUND OF THE INVENTION

1. Field of the Invention

The present invention relates to novel hernia repair mesh prostheses, and to methods of using same to repair hernias. More particularly, the present invention relates to such prostheses which drastically reduce hernia recurrences and which are relatively simple to construct and install, and to methods of using same.

2. Background Art

Hernias are common medical problems involving a protrusion of an organ or body part through a rupture in connective tissue or through a wall of a cavity in which is normally enclosed. Some known types of hernias are in the groin area, including direct, indirect and femoral hernias, and others are in the abdominal wall.

Medical devices and procedures for repairing hernias are widely known, and have been reported well over one hundred years ago, and treatment of hernias has gone through incremental improvements over time, including the early work of A. P. Cooper, The Anatomy And Surgical Treatment Of Abdominal Hernia In Two Parts, London, Longman, Rees, Orme, Brown and Green 1827, the landmark advancement known as the Lichtenstein repair (I L Lichenstein, A G Schulman, P K Amid, M M Montilor, The Tension Free Hernioplasty, AnJ Surgery, 1989; 157:188-193, the placement of a truss-like mechanical support in the preperitoneal space utilizing the principals of Pascal's Law, R E Stoppa, J L Rives, C R Warlaumont, et al., The Use Of Dacron In The Repair Of Hernias Of The Groin, Surg. Clin. North Am, 1984; 64:269-285, and placement of mesh posterior to the muscles and investing fascia of the abdominal wall such that the kinetic force of abdominal pressure is used in a reverse role—instead of causing a hernia, the force is used to support the repair, L M Nyhus, R E Condon, H N Harkins, Clinical Experience With Preperitoneal Hernial Repair For All Types Of Hernias Of The Groin, Am J Surg, 1960; 100:234-244.

Recently, attempts are being made to classify hernias with the idea of standardization of repair for measurement of postoperative outcomes, while the concept of “individualization of repair” related to operative and anatomical findings is the foundation for modern day hernioplasties. L M Nyhus, Individualization of Hernia Repair: a New Era, Surgery, 1992; 114:1. To date, no single technique employed for hernioplasty can be performed for all the classifications of hernia within the groin, but instead individualized hernioplasties are required for variability of location of hernia and size hernia sac and defects, as well as other associated complications of incarceration and strangulation. Id.

See also, the discussion set forth in N. S. Tyagi et al., A New layered Preperitoneal Replacement of the Ilipubic Tract By an Anatomically Analogous Bilaminar Mesh Prosthesis For Inguinal and Femoral Herniorrhaphy, and in N. S. Tyagi, Minimally Invasive Bilaminar Mesh Groin Herniorrhaphy, presented at XXXII World Congress of the International College of Surgeons, Singapore, 2000. The full disclosure of the above-identified papers by N. S. Tyagi et al. and N. S. Tyagi are related to the present invention, and is incorporated herein by reference.

The effectiveness of any hernia repairing surgery is measured by not only the initial result thereof, but also by the avoidance of complications caused by the surgery, and ultimately by the minimization/absence of recurrence of the repaired hernias. Some complications associated with use of conventional mesh prostheses include shrinkage, migration and erosion of adjacent tissue by the prostheses, as well as formation of seroma, which is collection or trapping of body fluids near the repaired area and may lead to infection.

Of course, progress has been made in treatment of hernias over the years both in terms of the surgical procedures and in the technical advancement of prostheses used in the procedures. Unfortunately, even with the significant advancements over the years, recurrence still remains a very significant problem. This is so even with the shift towards individualization of hernia repair, which complicates the overall treatment procedure.

Thus, a need still exists for an improved hernia repair prosthesis. In particular, there is a need for such a prosthesis which can greatly reduce or eliminate recurrence, and for a relatively simple procedure for repairing hernias using the prosthesis.

### SUMMARY OF THE INVENTION

It is an object of the present invention to provide a hernia repair prosthesis which fills the discussed need in the art.

Another object of the present invention is to provide such a prosthesis which is relatively inexpensive to manufacture.

Yet another object of the invention is to provide such a prosthesis which can provided in a very compact form, and which can be easily manipulated and installed in a minimally invasive procedure.

Still another object of the invention is to provide such a prosthesis which can be widely used for repairing essentially all types of hernias, with only a limited number of sizes and shapes of the prosthesis.

Yet another object of the invention is to provide a small number of hernia repair methods which can be used for repairing a wide variety of hernias.

According to the invention, a hernia repair prosthetic element is provided comprising: a central portion which covers a hernia opening within a patient; plural support portions integrally connected about a periphery of the central portion for securing the central portion in covering relation to the hernia opening; the prosthetic element, including the central and support portions, being a substantially planar member formed of flexible mesh material; the support portions being foldable relative to the central portion so that the prosthetic element can be selectively manipulated between folded and unfolded shapes; said support portions being adapted to securely engage various components of the patient's body surrounding the hernia opening; and openings formed through said central portion for passage of fluid therethrough.

Preferably, the prosthetic element will come in only two basic shapes, one for use in relation to all groin hernias and one for use in relation to all abdominal hernias, although each of the two basic shapes will come in a couple different sizes to accommodate different size hernia openings and different size persons. It is also preferred that the prosthetic element will include securing members to initially support the element in place within the patient's body while the supporting portions are manipulated into engagement with the various portions of the patient's body surrounding the hernia opening, the securing members including either dissolvable hooks projecting from the element and/or a small number of sutures bonding the central portion in proximity to the hernia opening.

Still further, it is preferred that the flexible mesh material may be formed of propylene or other suitable materials, the central portion will have an enhanced strength, such as being formed of two layers of the mesh material, and for the prosthetic element used to repair abdominal hernias, the central portion will also be stretchable to absorb greater forces imposed thereon.

For a more complete understanding of the present invention, the reader is referred to the following detailed description section, which should be read in accordance with the accompanying drawings. Throughout the following description, like numbers refer to like parts.

### BRIEF DESCRIPTION OF THE DRAWINGS

FIG. 1 is a plan view of a hernia repair mesh prosthesis in connection with a first embodiment of the invention, particularly related to repair of groin or femoral or inguinal hernias;

FIG. 2 is a plan view of a hernia repair mesh prosthesis in connection with a second embodiment of the invention, particularly related to repair of abdominal or ventral hernias;

FIG. 3 is a side elevational view of the hernia repair mesh prosthesis of FIG. 2; and

FIG. 4 is a magnified, sectional view of a portion of the hernia repair mesh prosthesis of FIG. 2.

### DETAILED DESCRIPTION OF THE PRESENTLY PREFERRED EMBODIMENTS

First Embodiment

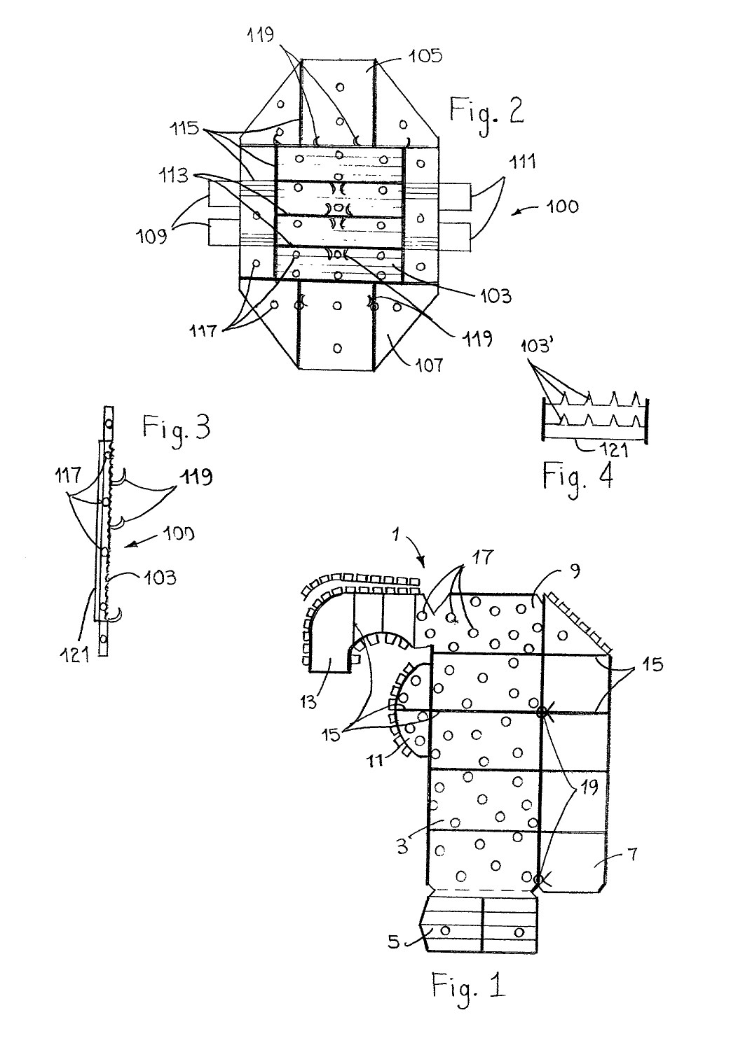

Referring now to FIG. 1, a plan view of a hernia repair mesh prosthesis in connection with a first embodiment of the invention, particularly related to repair of groin or femoral hernias, is shown generally at 1. The prosthesis 1 is a substantially planar member formed of polypropylene mesh or other appropriate material(s), and is universally suitable for herniorrhaphy for all levels of groin hernias, including direct, indirect, sliding, femoral and recurrent hernias. Throughout the present specification, a number of relative positional terms such as upper, lower, and side are used to illustrate parts of the prosthesis 1 in the orientation thereof shown in the drawings. These terms are used for purposes of illustration rather than limitation, and as such, are not intended to be fixed and absolute. The reader will realize that the prosthesis could be inverted, folded, or placed in a different orientation than the one shown, and that after such modification, these relative terms would no longer apply.

The prosthesis 1 preferably includes a main, central substantially rectangular portion 3, which is provided for supportively covering the hernia opening or rupture to be repaired. Particularly, the main central portion 3 of the prosthesis is sized such that it supports the entire orifice of Fruchaud, covering all areas of potential weakness. The prosthesis 1 further includes a number of support portions 5, 7, 9, 11 and 13, respectively, integrally and sequentially connected around the periphery of the central portion 3. More specifically, the prosthesis 1 in this embodiment includes a lower support portion 5 attached to the bottom of, and having approximately the same width as, the central portion 3. A primary side support portion 7 is also provided attached to one side of the central portion 3. The primary side support portion 7 has substantially the same height as the central portion 3. An upper support portion 9, having about the same width as the central portion, is attached to the top thereof, and a triangular panel 8 interconnects the primary side support portion 7 to the upper support portion 9. The triangular panel 8 has a plurality of tabs 10 extending outwardly from an outer edge thereof. The prosthesis also includes a downwardly curved upper side extension 13 attached to the upper support portion opposite the triangular panel, and a D-shaped side support portion 11 attached to the central portion 3 a short distance below the curved upper side extension. Both the curved upper side extension 13 and the D-shaped side support portion 11 also have a plurality of tabs attached thereto and extending outwardly thereon.

The prosthesis 1 also includes a plurality of reinforcement ribs 15 formed as part of the central portion and the adjoining support portions, to provide strength to the prosthesis. Indicated at 17 are relatively large openings, e.g., having diameters in a range of 1-7 mm, formed in selected areas of the prosthesis for permitting drainage, or fluid flow, from one side of the prosthesis to the other. These openings are provided in the central portion 3 as well as in at least some of the support portions, such as the upper support portion 9 and the D-shaped side support portion 11. The openings 17 desirably permit fluid drainage or flow from one side of the prosthesis to the other, and thereby function to minimize or eliminate seroma formation, or the collection of fluid on one side of the prosthesis, which could otherwise lead to infection or other complications.

It is preferred that those portions of the prosthesis having the drain holes 17 formed therein will have an enhanced strength as compared to the other portions of the prosthesis, in order to compensate for the reduction in strength caused by the placement of the drain holes therein, and because it is the portion that functionally closes the hernia opening. For example, the enhanced strength portions may be formed with two layers of the polypropylene mesh, while some of the other portions may be formed with only a single layer. Such multiple layer construction is currently preferred because of limitations in the strength of available materials. Once stronger materials are developed, however, it would be preferred to use a single layer for the entire prosthesis because the single layer device would be easier to construct and less likely to inhibit fluid drainage. Of course, all portions of the prosthesis 1 may be formed with multiple mesh layers and/or with the openings 17 formed therein.

With the above described construction, the prosthesis 1 is desirably effective to eliminate all areas of weakness within the myopectineal orifice, as well as to provide an adequate mechanism for transfer of intra-abdominal pressure, the kinetic force for herniation.

According to an important aspect of the invention, the prosthesis 1 may be universally applied for the repair of essentially all groin hernias, so that it may be economically produced in large quantities, and so that it is not necessary for hospitals, etc. to maintain a large inventory of different types of prostheses. Particularly, the prosthesis 1 can be used for either left or right side hernias by simply, reversibly orienting it in either direction, while the prosthesis will be available in a limited number of different sizes sufficient to be used for all size groin hernias. For example, some typical sizes for the main central portion 3 are 5 cm height×7 cm width; 6 cm height×8½ cm width; and 7½ cm height×10 cm width.

Once surgically installed in a patient's myopectineal orifice relative to the hernia, the support portions 5, 7, 9, 11 and 13, respectively, function to simply yet reliably support the prosthesis in place, in cooperation with intra-abdominal force, which is based on physical principles and the layered anatomy of the myopectineal orifice. Particularly, the support portion 5 is adapted to be folded or bent behind a bone in the groin region near the hernia, the support portion 7 is adapted to be folded or bent behind the rectus muscle near the hernia, the support portion 9 is adapted to be bent or folded behind the transversus muscle, the support portion 11 is adapted to be bent or folded behind the internal inguinal ring, and the support portion 13 is adapted to extend around the spermatic cord structures, as discussed further below in relation to methods of hernia repair according to the invention.

Although the five support portions 5, 7, 9, 11 and 13 are shown and preferred, it will be understood that a different number of supports and/or differently sized and shaped supports could be used to fit a specific application.

### Method of Hernia Repair Using Prosthesis According to the First Embodiment

A method of repairing a groin hernia (groin herniorrhaphy) using the prosthesis 1 preferably involves steps of: folding the several support portions 5, 7, 9, 11 and 13 upon the main, central portion 3 to form a thin, fan shaped, compact member; administering the folded member within the patient's body by inserting same through a small incision (e.g., 3-5 cm long) in the body using a scope; unfolding the support portions from the main portion; securing the prosthesis to the Cooper's ligament extending up the femoral vein in the preperitoneal space using by one or two sutures 19 applied at the boundary between the main portion 3 and the support portion 7; further securing the main portion in place using the several support portions 5-13; and removing the scope and closing the incision. Optionally, the securing step involving sutures can be modified by providing dissolvable hooks (such as those discussed in relation to FIGS. 2-4 below) integrally with the prosthesis at substantially the same locations as the sutures 19 would otherwise be applied. The dissolvable hooks, formed of polyglycolic acid or other suitable materials, are easily attached to body tissue for holding the prosthesis in place, and then dissolve after one month or so.

More specifically in relation to placement of the support portions 5, 7, 9, 11 and 13, after the prosthesis 1 is secured to the Cooper's ligament using the sutures 19 or the dissolvable hooks, the following steps occur: the remnant of the iliopubic tract is interposed between the two laminae, allowing the internal inguinal ring to return to its normal anatomical location; the anterior folded support member 13 is secured to the shelving edge of the inguinal ligament, extending up to the femoral artery, external to the iliopubic tract; both of the support portions 7, 9 were placed in the preperitoneal space, and anchored to the posterior surface of RAM with a cuff of at least 2 cm from the lateral border. Superiorly, both portions were secured to the posterior surface of the TAM, extending about 3 cm from the inferior border of the IOM. Laterally, the upper support portions of the prosthesis extend about 3-4 cm from the internal, inguinal ring, extending up to the psoas muscle. The inferior border of the IOM was approximated to the prosthesis just medial to the internal ring, assuring placement of the internal ring posterior to the IOM.

Such surgical method is minimally invasive, typically can be performed in approximately 30-45 minutes, and with less than 50 cc of blood loss. Moreover, such surgical procedure has thus far proven to have eliminated instances of hernia recurrence.

Very desirably, the support portions 5, 7, 9, 11 and 13, together with the sutures 19 or dissolvable hooks, function to very reliably support the prosthesis in place even in the event of severe coughing or other forceful actions by the patient subsequent to surgery. With conventional prosthetic devices (or plugs) and procedures, a significant problem that sometimes occurs is migration of the prosthetic devices from the area of the rupture. This not only results in recurrence of the hernia, but is also very uncomfortable for the patient.

Also very desirable is the fact that the prosthesis 1 of the invention is reversible, for use in relation to left or right groin hernias, and can be used for repairing all types of groin hernias. Again, however, the prosthesis would be available in a few standard sizes, but inventory of the prosthesis could be kept to a minimum, contrary to conventional practices.

Second Embodiment

Referring now to FIGS. 2-4, there is shown a hernia repair mesh prosthesis 100 according to a second embodiment of the invention, particularly related to repair of abdominal wall hernias or ventral hemiorrhaphy. FIG. 2 is a plan view of the prosthesis 100, FIG. 3 is a side elevational view of same, and FIG. 4 is an enlarged, sectional view of a stretchable portion of same. Like the prosthesis 1 of the first embodiment, the prosthesis 100 is a substantially planar member formed of polypropylene mesh or other appropriate material(s), although it is distinct from the prosthesis 1 in several significant aspects.

The prosthesis 100 preferably includes a substantially rectangular, main, central portion 103 which actually covers the hernia rupture to be repaired, and a number of support portions or flaps 105, 107, 109 and 111 integrally connected around the periphery of the central portion 103. The prosthesis 100 further includes suture type reinforcement ribs 113 extending through the central portion 103, other reinforcement ribs 115 at ends of the central portion and extending through support portions 105, 107, dissolvable hooks 119, and a dissolvable sheet 121. As in the prosthesis 1 of the first embodiment, relatively large openings 117 (having diameters of 1-7 mm) are provided in the central and support portions of the prosthesis 100 for permitting drainage or fluid flow from one side of the prosthesis to the other, and at least the central portion 103 of the the prosthesis 100 is preferably constructed of two layers of polypropylene mesh connected or laminated one on top of the other for enhanced strength. The openings 117 are provided over the entire main portion 103, as well as most of the support portions of the prosthesis.

One significant difference from the first embodiment is the particular construction of the central portion 103. As shown in FIGS. 2-4, the polypropylene mesh in the central section 103 is generally flat in its normal orientation, but it is actually formed with a number of folds 103′ in each of the polypropylene layers such that the central portion 103 is resiliently, stretchably expandable to some extent. Each of the folds 103′ extend widthwise of the prosthesis 100 for the full width of the central portion 103 and has a small width of 1-2 mm, for example, in the longitudinal direction of the prosthesis. The folds 103′ desirably permit the central portion 103 to expand or stretch to a substantial extent in situations where excess force is applied thereto, e.g., when the patient coughs, for thereby absorbing the excess force, and after the excess force is absorbed, the central portion reverts to its normal substantially planar shape under the resiliency of the central portion. The resiliently stretchable/expandable nature of the central portion 103 is very desirable for absorbing the larger forces experienced in the abdomen, in comparison to those experienced in the groin region, and assuring that the element 100 remains properly in place within the patient. As shown, the openings 117 extend through the folds 103′ of the central portion 103, as well as through the adjacent support portions 105-111.

Another significant difference from the first embodiment is the use of numerous dissolvable hooks 119 on the prosthesis 100 and projecting from the face of the prosthesis. In surgically fixing the prosthesis within a patient, the support portions 105-111 are simply slid behind and/or between the adjacent muscle tissue surrounding the hernia opening, or more specifically between the muscle and the peritoneum, and the hooks 119 secure the prosthesis in place for approximately one month, until they dissolve, but by which time the patient's body has grown around the prosthesis to some extent.

Still another significant difference from the first embodiment is the presence of the dissolvable sheet 121 on the lower face of the prosthesis covering the area corresponding to the central section 103. Like the hooks 119, the sheet may be formed of polyglycolic acid or other appropriate material which dissolves after a short period of one month or so. Use of the sheet 121 prevents undesirable adhesion and bonding between the central section 103 and the body muscle and/or tissue surrounding same.

### Method of Hernia Repair Using Prosthesis According to the Second Embodiment

A method of repairing an abdominal hernia (ventral or inguinal herniorrhaphy) using the prosthesis 100 is similar to the method described above for repairing a groin hernia using the prosthesis 1. Preferably, the method involves steps of: folding the several support portions 105-111 upon the main, central portion 103 to form a thin compact member; administering the folded member within the patient's body by inserting same through a small incision using a scope; unfolding the support portions from the main portion; securing the main portion in place using the dissolvable hooks 119; further securing the prosthesis 100 in place by inserting the support portions 105-111 between the muscle and peritoneum; and removing the scope and closing the incision.

Again, such surgical method is minimally invasive, typically can be performed in less than one hour and with minimal blood loss. Moreover, such surgical procedure has also, thus far, proven to have eliminated instances of hernia recurrence. This is very significant because conventionally there has been a rather large recurrence rate of approximately 50% for abdominal wall hernias.

Also, like the first embodiment, the prosthesis 100 has the same basic or standard form for all abdominal wall hernias, but would be available in a few standard sizes for desirably minimizing the variety and amount of inventory of the prosthesis 100 which would be required to be kept available at hospitals, etc. For example, some typical sizes for the central section are 4 cm height×3 cm width; 8 cm height×5 cm width; 10 cm height×7 cm width; and 12 cm height×10 cm width.

Although the present invention has been described herein with respect to preferred embodiments thereof, the foregoing description is intended to be illustrative, and not restrictive. Many modifications may be made to the described embodiments without departing from the scope hereof. For example, the exact shape of the prostheses shown according to the preferred embodiments need not be strictly maintained, while materials other than polypropylene mesh may be used in forming the prostheses.

I claim:

1. A hernia repair prosthetic element comprising:
a central portion which covers a hernia opening within a patient;
plural support portions integrally connected about a periphery of the central portion for securing the central portion in covering relation to the hernia opening;
the prosthetic element, including the central and support portions, being a substantially planar member formed of flexible mesh material;
the support portions being foldable relative to the central portion so that the prosthetic element can be selectively manipulated between folded and unfolded shapes;
said support portions being adapted to securely engage various components of the patient's body surrounding the hernia opening, respectively; and
said central portion has openings formed therethrough for passage of fluid.

2. The prosthetic element according to claim 1, further including a securing member provided with said prosthetic element to initially support said element within the patient's body while the support portions are manipulated into engagement with the various components of the patient's body surrounding the hernia opening.

3. The prosthetic element according to claim 2, wherein said securing member is a dissolvable hook projecting from the element.

4. The prosthetic element according to claim 1, wherein said element is formed of polypropylene mesh.

5. The prosthetic element according to claim 1, wherein said element is for use in repairing groin hernias, and has a standard shape suitable for repairing substantially all groin hernias.

6. The prosthetic element according to claim 1, wherein said support portions include a first support portion which is disposed around the patient's spermatic cord, a second support portion which engages the patient's rectus muscle, a third support portion which engages the patient's transverses muscle, a fourth support portion which engages the patient's internal inguinal ring, and a fifth support portion which engages a bone in the patient's groin area.

7. The prosthetic element according to claim 5, wherein said element is reversible for repairing right and left side groin hernias.

8. The prosthetic element according to claim 1, wherein said openings have a diameter in a range of 1-7 mm.

9. The prosthetic element according to claim 1, wherein said central portion includes means for enhancing the strength thereof.

10. The prosthetic element according to claim 9, wherein said strength enhancing means includes reinforcing ribs extending across the central portion.

11. The prosthetic element according to claim 9, wherein said strength enhancing means includes multiple layer construction of the central portion from a mesh sheet material.

12. The prosthetic element according to claim 1, wherein said element is for use in repairing abdominal hernias, and has a standard shape suitable for repairing substantially all abdominal hernias.

13. The prosthetic element according to claim 12, wherein said central portion is resiliently expandable.

14. The prosthetic element according to claim 13, wherein said central portion includes resiliently expandable folds of polymeric mesh material.

15. The prosthetic element according to claim 12, wherein said support portions surround the periphery of said central portion and are adapted to be disposed between the muscle tissue and peritoneum of the patient's body surrounding the hernia opening.

16. The prosthetic element according to claim 1, further including a sheet of dissolvable material provided on one face of said central portion.

17. A method of hernia repair using a substantially planar prosthetic element including a central portion which covers a hernia opening within a patient and plural support portions integrally connected about a periphery of the central portion for securing the central portion in covering relation to the hernia opening, comprising the steps of:
making a small incision in a patient's body in the vicinity of the hernia;
folding the support portions upon the central portion 3 in a thin, compact shape;
administering the folded member within the patient's body by inserting same through the small incision using a scope;
unfolding the support portions from the main portion;
engaging the support portions with various components of the patient's body surrounding the hernia opening such that the central portion covers the hernia opening; and
removing the scope and closing the incision.

18. The method of hernia repair according to claim 17, further including the step of initially securing said element within the patient's body while the support portions are manipulated into engagement with the various components of the patient's body surrounding the hernia opening.

19. The method of hernia repair according to claim 17, wherein said element is formed of polypropylene mesh, and said central portion has openings formed therethrough for passage of fluid.
